SEQUENCE LISTING (21 sequences)

SEQ ID NO: 1 - DNA, 28 bp, Artificial Sequence
  Description of Artificial Sequence Synthetic primer
gctactagaggaagagattcaaaatcct

SEQ ID NO: 2 - DNA, 27 bp, Artificial Sequence
  Description of Artificial Sequence Synthetic primer
atcaacatctaaatatactccgccaaa

SEQ ID NO: 3 - DNA, 32 bp, Artificial Sequence
  Description of Artificial Sequence Synthetic primer
atctcaaatcaataaacctacagtacctacaa

SEQ ID NO: 4 - DNA, 21 bp, Artificial Sequence
  Description of Artificial Sequence Synthetic primer
cagatgcagccaaagttgttg

SEQ ID NO: 5 - DNA, 26 bp, Artificial Sequence
  Description of Artificial Sequence Synthetic primer
tgttgccttatctcgaagtacaagtt

SEQ ID NO: 6 - DNA, 27 bp, Artificial Sequence
  Description of Artificial Sequence Synthetic primer
tatagatggtgtaagtttaggtgcagc

SEQ ID NO: 7 - DNA, 21 bp, Artificial Sequence
  Description of Artificial Sequence Synthetic primer
ttaggcgtgttttttggcaat

SEQ ID NO: 8 - DNA, 28 bp, Artificial Sequence
  Description of Artificial Sequence Synthetic primer
catatcctttcttctcctcttcttcttt

SEQ ID NO: 9 - DNA, 32 bp, Artificial Sequence
  Description of Artificial Sequence Synthetic primer
atttctaaccaaacatcagttatagattctca

SEQ ID NO: 10 - DNA, 24 bp, Artificial Sequence
  Description of Artificial Sequence Synthetic primer
cagcagtcatgggtgcagtagtag

SEQ ID NO: 11 - DNA, 28 bp, Artificial Sequence
  Description of Artificial Sequence Synthetic primer
agctgatatactatttccttgtgccaat

SEQ ID NO: 12 - DNA, 26 bp, Artificial Sequence
  Description of Artificial Sequence Synthetic primer
gcagtttctcctgcaattattgtacc

SEQ ID NO: 13 - DNA, 35 bp, Artificial Sequence
  Description of Artificial Sequence Synthetic primer
tgaattaattgttttagcaagaaataactcagtag

SEQ ID NO: 14 - DNA, 34 bp, Artificial Sequence
  Description of Artificial Sequence Synthetic primer
aaaaatattattaaatctgtttctccctcttcat

SEQ ID NO: 15 - DNA, 32 bp, Artificial Sequence
  Description of Artificial Sequence Synthetic primer
ttaaatctgtttctccctcttcataatgtaaa

SEQ ID NO: 16 - DNA, 400 bp, Clostridium difficile
gctactagaggaagagattcaaaatcctcaatttgataatatgaaattttacaaaaaaaggatggaatttatatatgatagacaaaaaaggtttataaattattataaatctcaaatcaataaacctacagtacctacaatagatgatattataaagtctcatctagtatctgaatataatagagatgaaactgtattagaatcatatagaacaaattctttgagaaaaataaatagtaatcatgggatagatatcagggctaatagtttgtttacagaacaagagttattaaatatttatagtcaggagttgttaaatcgtggaaatttagctgcagcatctgacatagtaagattattagccctaaaaaattttggcggagtatatttagatgttgat

SEQ ID NO: 17 - DNA, 350 bp, Clostridium difficile
cagatgcagccaaagttgttgaattagtatcaactgcattagatgaaactatagacttacttcctacattatctgaaggattacctataattgcaactattatagatggtgtaagtttaggtgcagcaatcaaagagctaagtgaaacgagtgacccattattaagacaagaaatagaagctaagataggtataatggcagtaaatttaacaacagctacaactgcaatcattacttcatctttggggatagctagtggatttagtatacttttagttcctttagcaggaatttcagcaggtataccaagcttagtaaacaatgaacttgtacttcgagataaggcaaca

SEQ ID NO: 18 - DNA, 280 bp, Clostridium difficile
ttaggcgtgttttttggcaatatatcctcaccagcttgttctgaagaccatgaggaggtcatttctaaccaaacatcagttatagattctcaaaaaacagaaatagaaactttaaatagcaaattgtctgatgctgaaccatggttcaaaatgaaagacgacgaaaagaaagctattgaagctgaaaatcaacgtaaagctgaagaagctaaaaaagctgaagaagctaaaaaggctgaagaacaacgcaaaaaagaagaagaggagaagaaaggatatg

SEQ ID NO: 19 - DNA, 200 bp, Clostridium difficile
cagcagtcatgggtgcagtagtaggagcagtttctcctgcaattattgtacctaaaatgcttaaattaatggaagaaggttatggtacagaaaaaagtattccacagatgcttttagcaggaacttctatagacgatattttcgtaattgtgatgtttacagtattcacaggattggcacaaggaaatagtatatcagct

SEQ ID NO: 20 - DNA, 135 bp, Clostridium difficile
tgaattaattgttttagcaagaaataactcagtagatgatttgcaagaaattttatttatgtttaagccattagtaaaaaaacttagtagagttttacattatgaagagggagaaacagatttaataatattttt

SEQ ID NO: 21 - DNA, 135 bp, Clostridium difficile
aaaaatattattaaatctgtttctccctcttcataatgtaaaactctactaagtttttttactaatggcttaaacataaataaaatttcttgcaaatcatctactgagttatttcttgctaaaacaattaattca

(19) United States
(12) Patent Application Publication
(10) Pub. No.: US 2011/0287965 A1
(43) Pub. Date: Nov. 24, 2011

(54) METHODS AND COMPOSITIONS TO DETECT CLOSTRIDIUM DIFFICILE

(75) Inventors: Tat-Kin Tsang, Winnetka, IL (US); Xiangwen Meng, Skokie, IL (US); Hongjun Zhang, Glenview, IL (US)

(73) Assignee: TZAM DIAGNOSTICS LLC, Wilmette, IL (US)

(21) Appl. No.: 13/130,846

(22) PCT Filed: Nov. 24, 2009
(86) PCT No.: PCT/US09/65723

§ 371 Date: Aug. 12, 2011

Related U.S. Application Data
(60) Provisional application No. 61/118,186, filed on Nov. 26, 2008.

(51) Int. Cl. C40B 30/04; C07H 21/04; C40B 30/00

(52) U.S. Cl. 506/9; 506/7; 536/24.32; 536/24.33

(57) ABSTRACT

Methods for detecting strains of Clostridium difficile (e.g. toxigenic and non-toxigenic) in a specimen are disclosed. Oligonucleotide primer pairs based on the sequences of multiple loci of the bacteria are disclosed. In one application, up to five loci in the genomic DNA sequences of Clostridium difficile were amplified. Two fragments of C. difficile were amplified from each locus, wherein a second fragment was an internal fragment of the first fragment.

### BACKGROUND

Microorganisms such as bacteria and viruses cause serious infectious diseases such as tuberculosis, cholera, hepatitis and meningitis. To diagnose and cure bacterial infections, rapid and early identification of specific disease causing pathogens in clinical specimens are required. Several bacterial infections do not show characteristic symptoms initially, which requires sensitive and specific tools to diagnose infections.

Clostridium difficile is a species of Gram-positive bacteria of the genus Clostridium. Clostridia are anaerobic, spore-forming rods (bacillus). C. difficile is a significant cause of pseudomembranous colitis. It is an infection of the colon, that often occurs after normal gut flora is damaged by use of antibiotics. As a result, the C. difficile that naturally reside in the body may overgrow. This overgrowth is harmful because the bacterium releases toxins that can cause bloating, constipation, and diarrhea with abdominal pain, which may become severe.

Some of the detection systems include for example, cytotoxicity assay, where C. difficile toxin detection is through its cytopathic effect in cell culture and neutralization with specific anti-sera. This assay is a standard for C. difficile detection. Toxigenic culture, in which organisms are cultured on selective medium and tested for toxin production, is slow and labor-intensive.

Diagnosis of C. difficile-associated diarrhea is primarily based on clinical symptoms and stool assays for presence of the pathogen or toxins. Because nontoxigenic isolates are not responsible for the diarrhea as they do not produce toxins, accurate testing becomes even more important for exploring the association of C. difficile with other intestinal ailments and helps to prevent unnecessary treatment while minimizing the overuse of antibiotics.

Therefore, sensitive and rapid detection systems are desired.

### SUMMARY

A method of detecting Clostridium difficile in a specimen includes determining the presence of one or more nucleic acid fragments representing a plurality of loci selected from the group consisting of nucleic acid regions whose sequences are designated as tcdA, tcdB, tcdC, tcdD and edu2. In an aspect, at least one of tcdC, tcdD and edu2 is tested.

In an aspect, the presence of the nucleic acid fragments are determined by amplification of the plurality of loci by a plurality of primers selected from the group consisting of nucleic acid sequences: GCTACTAGAGGAAGAGATTCAAAATCCT (SEQ ID NO: 1); ATCAACATCTAAATATACTCCGCCAAA (SEQ ID NO: 2); ATCTCAAATCAATAAACCTACAGTACCTACAA (SEQ ID NO: 3); CAGATGCAGCCAAAGTTGTTG (SEQ ID NO: 4); TGTTGCCTTATCTCGAAGTACAAGTT (SEQ ID NO: 5); TATAGATGGTGTAAGTTTAGGTGCAGC (SEQ ID NO: 6); TTAGGCGTGTTTTTTGGCAAT (SEQ ID NO: 7); CATATCCTTTCTTCTCCTCTTCTTCTTT (SEQ ID NO: 8); ATTTCTAACCAAACATCAGTTATAGATTCTCA (SEQ ID NO: 9); CAGCAGTCATGGGTGCAGTAGTAG (SEQ ID NO: 10); AGCTGATATACTATTTCCTTGTGCCAAT (SEQ ID NO: 11); GCAGTTTCTCCTGCAATTATTGTACC (SEQ ID NO: 12); TGAATTAATTGTTTTAGCAAGAAATAACTCAGTAG (SEQ ID NO: 13); AAAAATATTATTAAATCTGTTTCTCCCTCTTCAT (SEQ ID NO: 14); and TTAAATCTGTTTCTCCCTCTTCATAATGTAAA (SEQ ID NO: 15).

The presence of the nucleic acid fragments is determined by amplification of the plurality of loci representing at least two independent loci of C. difficile.

In an aspect, the nucleic acid fragments representing each locus are amplified by one forward and two reverse primers, wherein one of the reverse primers is internal to the other reverse primer.

Suitable specimens are selected from the group consisting of a tissue biopsy, stool, swab, and bodily fluids. In an aspect, the specimen is isolated DNA. In an aspect, the amplification reaction is performed directly on the specimen.

The presence of one or more nucleic acid fragments representing the plurality of loci is also determined by hybridization with one or more probes.

In an aspect, the nucleic acid fragments are simultaneously amplified.

In an illustrated embodiment, a novel one-step multiplex polymerase chain reaction detection system using a plurality of genetic loci of Clostridium difficile was developed to identify Clostridium difficile present in clinical specimens. In one application, up to five loci in the genomic DNA sequences of Clostridium difficile were chosen as amplification targets. Two fragments of Clostridium difficile were amplified from each locus, wherein a second fragment was an internal fragment of the first fragment.

A method of detecting Clostridium difficile in a specimen includes:
(a) performing a multiplex polymerase chain reaction, wherein a plurality of DNA fragments representing a plurality of loci in Clostridium difficile are amplified by a plurality of primers, where for each pair of forward and reverse primers, one primer is internal to the other; and
(b) determining that a specimen is positive for Clostridium difficile if a number of amplified fragments is sufficient to detect the bacteria from other bacteria.

The multiplex polymerase chain reaction may be performed with an isolated Clostridium difficile DNA, wherein the Clostridium difficile DNA is isolated from a clinical specimen. The clinical specimen is selected from a group of gastrointestinal tract tissue, stool, urine, blood, saliva, mucus secretions, dental plaque, and other tissues capable of containing the bacteria. The multiplex polymerase chain reaction is also suitably performed directly on a biological sample or a specimen.

In an embodiment, a diagnostic kit to detect toxigenic and nontoxigenic strains of Clostridium difficile in a specimen includes in discrete containers:
(a) a plurality of primers to amplify a plurality of DNA fragments from the Clostridium difficile genomic sequence; and
(b) reagents to perform a multiplex polymerase chain reaction.

The diagnostic kit to detect Clostridium difficile also includes a DNA polymerase, nucleotides, and buffers. The plurality of primers in the diagnostic kit are capable of amplifying a plurality of DNA fragments so that half of the amplified fragments are internal to the other half of the amplified fragments.

In an embodiment, a method for detecting toxigenic and nontoxigenic strains of Clostridium difficile in a specimen includes:
(a) performing a multiplex polymerase chain reaction, wherein a first set of primers amplifies a first set of DNA fragments from a Clostridium difficile genomic sequence and a second set of primers amplifies a second set of DNA fragments from the Clostridium difficile genomic sequence that are internal to the first set of DNA fragments; and
(b) determining that a specimen is positive for toxigenic Clostridium difficile if at least fifty percent of all the fragments or at least two fragments representing either the first set or the second set of DNA fragments and the corresponding internal fragments are amplified.

In an embodiment, a method for detecting toxigenic and nontoxigenic strains of Clostridium difficile in a specimen includes:
(a) performing a multiplex polymerase chain reaction, wherein
   (i) a first DNA fragment from a Clostridium difficile genomic sequence is amplified by a first primer pair and an internal segment of the first DNA fragment is amplified by a second primer pair,
   (ii) a second DNA fragment from a Clostridium difficile genomic sequence is amplified by a third primer pair and an internal segment of the second DNA fragment is amplified by a fourth primer pair; and
(b) determining that a specimen is positive for toxigenic Clostridium difficile if at least one of the fragments and its internal fragment or fifty percent of all the fragments are amplified.

In an embodiment, a multiplex polymerase chain reaction with the first set of primers was performed separately from the multiplex polymerase chain reaction with the second set of primers. The first set of primers and the second set of primers amplified a total of ten fragments representing five loci in the Clostridium difficile sequence.

In an embodiment, a Clostridium difficile locus is selected from a group of coding, non-coding, exons, introns, and regulatory regions. A plurality of loci to be amplified was selected from a group of DNA sequences that include tcdA, tcdB, tcdC, tcdD and edu2, to identify toxigenic and nontoxigenic strains of Clostridium difficile at the same time.

In a multiplex PCR disclosed herein, the first primer pair and the second primer pair have a common primer per locus. A multiplex polymerase chain reaction to detect Clostridium difficile was performed, wherein up to ten DNA fragments representing five C. difficile loci were amplified by fifteen primers, the ten DNA fragments representing five internal fragments. A set of fifteen primers comprise five forward and ten reverse primers.

In an embodiment, a method of detecting toxigenic and nontoxigenic strains of Clostridium difficile in a specimen includes:
(a) designing a plurality of primers in a plurality of loci of Clostridium difficile, wherein the primers
   (i) are specific for Clostridium difficile;
   (ii) have a low frequency of mutations in primer binding sites;
(b) performing a multiplex polymerase chain reaction wherein the plurality of primers amplify a plurality of DNA fragments representing the plurality of loci in Clostridium difficile; and
(c) determining that a specimen is positive for Clostridium difficile if at least fifty percent of all the DNA fragments or at least four DNA fragments representing two loci in Clostridium difficile are amplified or by other empirically or statistically derived criteria.

In an embodiment, a diagnostic kit to detect toxigenic and nontoxigenic strains of Clostridium difficile in a specimen includes in discrete containers:
(c) a first set of primers to amplify a first set of DNA fragments from a Clostridium difficile genomic sequence;
(d) a second set of primers to amplify a second set of DNA fragments from Clostridium difficile genomic sequence that are internal to the first set of DNA fragments; and
(e) reagents to perform a multiplex polymerase chain reaction.

The diagnostic kit to detect toxigenic and nontoxigenic strains of Clostridium difficile also includes a DNA polymerase, deoxynucleotides, and buffers. The kit includes a set of primers whose nucleotide sequences are listed herein. The kit may also include reagents: a buffer comprising 100 mM Tris-HCl (pH 8.3), 500 mM KCl, 16 mM MgCl₂, 0.01% (weight/volume) gelatin; and 10 mM of each deoxynuclotide. The concentration of primers is about 1.0 μM. A multiplex polymerase chain reaction is performed in a single reaction chamber, e.g., a single tube, in particular when in a kit.

A method for detecting toxigenic and nontoxigenic strains of Clostridium difficile in a specimen includes:
(c) performing a multiplex polymerase chain reaction, wherein a plurality of primers amplify a plurality of Clostridium difficile nucleic acid fragments are selected from the group consisting of nucleic acid molecules whose sequences are designated tcdA, tcdB, tcdC, tcdD and edu2; and
(d) determining that a specimen is positive for Clostridium difficile if a number of amplified fragments is sufficient to detect the bacteria in the specimen.

A plurality of nucleic acid fragments comprise a subset of a plurality of internal fragments. The plurality of Clostridium difficile nucleic acid fragments is selected from the group that includes RNA, cDNA, and genomic DNA.

Locus: location of a DNA sequence in a chromosome that encodes one or more products.

Multiplex PCR: A variant of conventional polymerase chain reaction that uses at least two or more primer pairs to amplify different stretches of a target DNA molecule simultaneously.

Nested PCR: A modified polymerase chain reaction that uses one or more primers (“nested”) whose sequences are complementary to an internal site of a DNA molecule that has been amplified with other primers.

Nucleic acid: includes DNA, RNA, cDNA, genomic DNA, natural, synthetic, nucleic acid analogs, and the like.

Oligonucleotide: single stranded DNA molecule with any length ranging from four to about 100 nucleotides.

Primers: Oligonucleotides of about 6 by to about 50 by in length used for initiating polymerase chain reaction.
Forward: a primer that may bind to one of the two complementary anti-parallel DNA strands.
Reverse: primer that may bind to a strand that is complementary to the strand to which the forward primer binds.

Specimen: A biological sample such as saliva, stools, urine, blood, biopsy, gastrointestinal tissue, tumor cells, mucus secretions, dental plaque, and other biological tissues, hospital samples, test swabs, culture plates, blood agar plates, meat products, food products, and environmental samples such as soil, water.

### BRIEF DESCRIPTION OF THE DRAWINGS

FIG. 1 shows an image of the agarose gel electrophoresis for individual primer pairs for test primers and PCR conditions.

FIG. 2 shows primer pairs combined into two sets for Multiplex PCR to test primer and M-PCR conditions, for specimens 630B, M6SB1, 630BB and M65BB.

FIG. 3 shows primer pairs combined into two sets for Multiplex PCR to test different DNA concentrations (1 ng, 0.5 ng, 0.25 ng, 0.125 ng, 0.0625 ng and 0.03125 ng).

FIG. 4 shows primer pairs combined into two sets for Multiplex PCR to test DNA concentrations in the picogram (pg) ranges.

FIG. 5 shows primer pairs combined into two sets for Multiplex PCR to test DNA concentrations at 100-0.5 pg levels.

FIG. 6 shows primer pairs combined into two sets for Multiplex PCR for specimen 1.

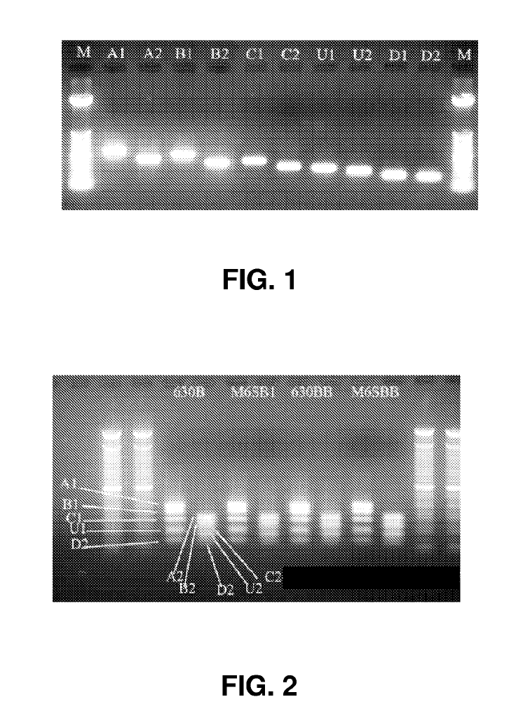

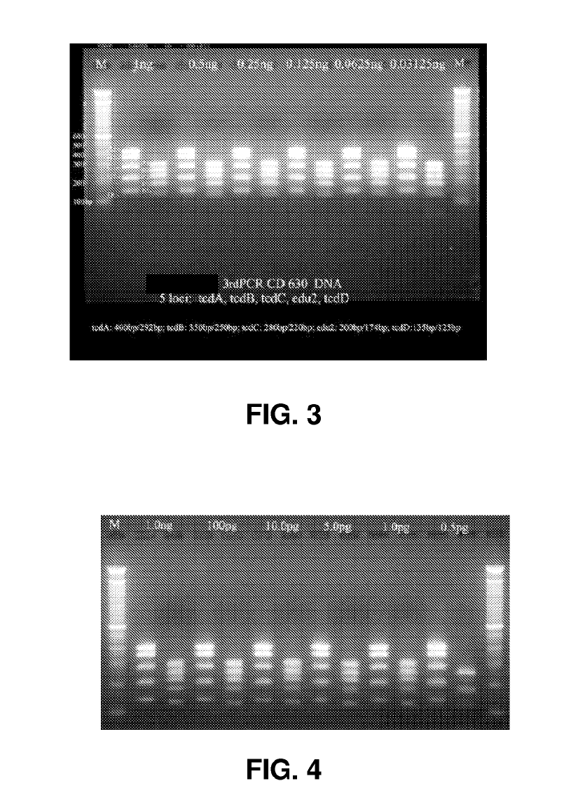

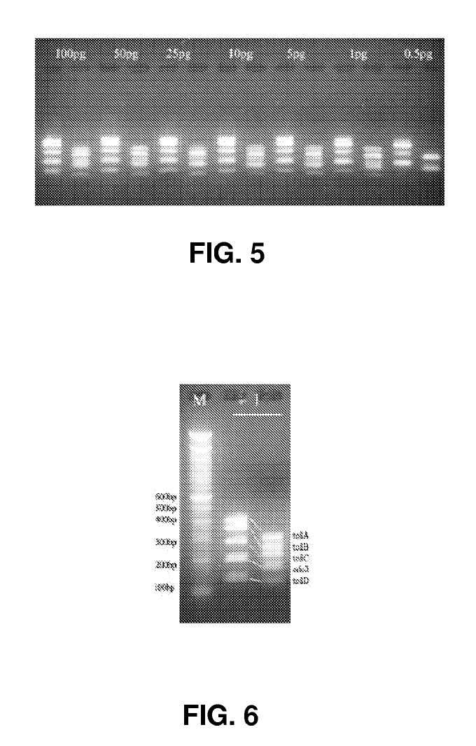

### DETAILED DESCRIPTION OF THE DISCLOSURE

In an embodiment, multiplex PCR was used to amplify 10 DNA fragments at the same time to identify tcdA, tcdB, tcdC, tcdD and edu2 genes or gene fragments in the Clostridium difficile genome. In an embodiment, amplifying selected 5 gene loci simultaneously increased sensitivity because the chances of amplifying several selected DNA regions are much higher than the chances of amplifying only one region. It also increased specificity, as probes for different loci can distinguish one pathogen from another. Each amplicon of the same loci serves as an internal control to exclude false-positives. The multiplex PCR method is sensitive and specific. It can detect C. difficile and determine its toxin genes at the same time, clinical identification of Clostridium difficile is possible.

In an embodiment, at least 2 or 3 or 4 or 5 fragments of the C. difficile target genes are amplified in a multiplex fashion. In an embodiment, at least 2 or more gene loci are amplified simultaneously but in separate reaction vessels. In an embodiment, instead of simultaneous amplification, 2 or more gene loci are amplified separately and after performing the first reaction.

A plurality of DNA fragments representing a plurality of loci from a bacterial genome is amplified using nested primers in a multiplex PCR reaction to positively identify the bacterial species. A plurality of DNA fragments from a specimen containing bacteria is amplified by a plurality of primers such that half of all the amplified fragments are internal to the other half.

In one example, up to five genetic loci from C. difficile genome are amplified using a set of at least five forward primers and ten reverse primers such that five of the amplified fragments are internal to the other five amplified fragments. A specimen is considered positive if at least five out of the ten amplicons are amplified or at least two of the five loci have both of their amplicons amplified. The amplified fragments can be resolved in a standard agarose gel electrophoresis or can be quantified using any other techniques such as real time quantitative PCR.

The number of loci to be selected for amplification depends on sequence similarity of C. difficile to other commonly occurring bacteria, availability of non-conserved genomic regions in the target bacteria when compared to the commonly occurring bacteria, availability of conserved genomic regions among the various strains of the target bacterial pathogen, and technical and practical feasibility to accommodate multiple samples.

Two or more genetic loci from C. difficile genome are amplified using a set of at least two forward primers and four reverse primers such that two of the amplified fragments are internal to the other two amplified fragments.

In one example, a multiplex PCR, wherein five forward primers and five reverse primers representing five different C. difficile loci are mixed to react in one reaction system and the five forward primers and five nested primers are allowed to react separately in another system. Alternatively, the five forward primers and the ten reverse primers are allowed to react in a single reaction system.

A multiplex PCR amplification was performed with isolated C. difficile DNA from clinical specimens that included infected tissues. A multiplex PCR amplification was performed with isolated bacterial DNA from bacterial cell cultures. Direct amplification of a clinical specimen without further processing to isolate DNA is also possible.

Amplifying more than one region of a nucleic acid molecule at the same time overcomes false-negative results because the possibility to amplify all or some of the selected DNA region is considerably higher when multiple regions are used rather than a single region. The amplified internal DNA fragments are helpful in minimizing false-positives. False negatives can be picked up by the one-step multiple-nested PCR. Unless the entire selected loci scanned by the multiplex PCR are deleted or mutated, the presence of some amplified fragments acts as an internal control, suggesting that the reaction has not failed, and helping rule out a false-negative result.

A standard touchdown PCR program is employed to amplify C. difficile DNA fragments. Briefly, a touchdown PCR involves decreasing the annealing temperature by 1° C. every second cycle to a ‘touchdown’ annealing temperature, which is then used for about 10 cycles, to optimize annealing temperatures. The basic idea is that any differences in Tₘ between correct and incorrect annealing gives a 2-fold difference in product amount per cycle (4-fold per ° C.). Therefore, the correct product is enriched over any incorrect products.

A diagnostic kit to detect C. difficile includes in discrete containers, primers, whose nucleotide sequences are determined based on the criteria described herein, a suitable DNA polymerase such as, for example, Immolase™ (Bioline, London, UK), deoxynucleotides, buffers, and optionally a set of pre-amplified bacterial DNA fragments for comparison, and a control bacterial DNA.

A diagnostic kit to detect C. difficile includes a set of up to five forward primers representing up to five different bacterial loci and up to ten reverse primers (including five nested primers) representing the five loci.

Multiplex nested PCR was used to amplify a plurality of DNA fragments from a plurality of loci at the same time to identify Clostridium difficile. Improved PCR sensitivity and specificity are due to selected amplification of various nested DNA regions. An internal control for each amplicon enhances identification of false negatives because amplification of some fragments indicates that the multiplex reaction has not failed.

To balance the primer mixture, in an embodiment, all primers were partitioned in two ways: a system in which all the primers were mixed together (system 1), and system 2 where five forward primers were mixed with either one set of reverse primers (system 2) separately (e.g. FIG. 6). System 1 amplified 10 DNA fragments in each tube at the same time, wherein system 2 amplified 5 DNA fragments in each tube.

Two or more genetic loci from C. difficile genome were amplified using a set of at least two forward primers and four reverse primers such that two of the amplified fragments are internal to the other two amplified fragments.

Up to five genetic loci from C. difficile genome were amplified using a set of at least five forward primers and ten reverse primers such that five of the amplified fragments are internal to the other five amplified fragments. A specimen is considered positive for C. difficile if at least five out of the ten amplicons illustrated herein are amplified or at least two of the five loci have both of their amplicons amplified (for example tcdA, tcdB, tcdC, tcdD and edu2 genes or gene fragments).

A multiplex nested PCR amplification was performed with isolated C. difficile DNA from clinical samples.

A diagnostic kit to detect C. difficile includes in discrete containers, primers, whose nucleotide sequences are described herein, a suitable DNA polymerase such as, for example, Immolase™ (Bioline, London, UK), deoxynucleotides, buffers, and optionally a set of pre-amplified C. difficile fragments for comparison, and a control C. difficile DNA. The diagnostic kit may also include a set of up to five forward primers representing five different C. difficile loci and 10 reverse primers (including 5 nested primers) representing the five loci.

In one aspect, the diagnostic kit to detect C. difficile includes a set of at least two forward primers representing two different C. difficile loci and four reverse primers (including 2 nested primers) representing the two loci.

The sensitivity of the one-step multiple-nested PCR assay (multiplex-nested-PCR) was investigated through a 36-40 cycle amplification of different dilutions of C. difficile DNA. The multiplex PCR method disclosed herein, was able to detect 10 DNA bands (in two tubes) in a single reaction when the DNA of C. difficile from the template was diluted to as little as 1.0 pg.

Amplified DNA fragments from a specimen containing C. difficile are confirmed with the one-step multiple-nested PCR because the disclosed primers produce two fragments for each of the five loci, one internal to the other. When both fragments are present for each locus, the DNA sample is inferred to be from C. difficile. On the contrary, if only one band is present for each locus, it may be caused by polymorphism in the amplified region.

A traditional PCR diagnosis for C. difficile is considered positive when one of two biopsied specimens from each part of the stomach is positive using two sets of primers derived from different genes, the disclosed one-step multiple-nested PCR assay was considered positive for C. difficile if 5/10 fragments or both DNA fragments from 2/5 loci were amplified.

The multiplex PCR system described herein is a sensitive and a specific method for identifying C. difficile in a clinical specimen.

The PCR amplification may be carried out in separate tubes or in a single tube for each loci involving two fragments. The 5 loci can all be amplified simultaneously in a single reaction vessel or in separate reaction vessels. In an embodiment, at least 3 or 4 or 5 loci are amplified simultaneously in a multiplex PCR. In an embodiment, at least 2 loci are amplified simultaneously in a multiplex PCR in a single reaction vessel. In an embodiment, the multiplex PCR is nested for each loci tested.

In an embodiment, about 50% of the fragments are amplified. These fragments may represent all of the loci tested or two fragments per loci for at least 2 loci that were tested. For example, an amplification produces at least one fragment representing 3 loci or 4 loci or 5 loci. For example an amplification produces at least 2 or 3 or 4 or 5 loci in which each loci has two fragments.

### EXAMPLES

### Example 1

### Detection of C. difficile Using Nested Multiplex PCR Assay

In this system, five loci (tcdA, tcdB, tcdC, tcdD and edu2) in the Clostridium difficile genome were chosen as amplification targets. For each locus, two fragments, one internal to another, are expected to be amplified by this novel multiplex PCR assay from the Clostridium difficile (Strain 630/ATCC BAA-1382 and 90556-M6S/ATCC 9689) genomic DNA.

After 35-41 cycles of amplification, 10 expected DNA fragments in two tubes were obtained with our multiplex PCR detection system, and were displayed in two DNA ladders in the 2.5% agarose gel with ethidium bromide staining (tcdA: 400 bp/292 bp; tcdB: 350 bp/250 bp; tcdC: 280 bp/220 bp; edu2: 200 bp/174 bp; tcdD:135 bp/125 bp). The sensitivity of the PCR assay was investigated next with a serial dilution of Clostridium difficile's DNA, and all the 10 DNA bands are able to be detected at the same time with as little as 1.0 pg of DNA after 40 cycles of amplification.

The DNA of C. difficile and other bacteria were isolated according to known methods with some modifications disclosed herein.

Buffers and Solutions: Ethanol, Potassium acetate (5 M), TE (pH 7.6), Cell lysis buffer: 10 mM Tris-Cl (pH 8.0), 0.05M EDTA (pH 8.0), 0.5%(w/v) SDS, 20 μg/ml DNase-free RNase. Other enzymes and buffers include DNase-free RNase (4 mg/ml), Proteinase K (20 mg/ml), DNA from mammalian tissue containing Clostridium difficile and other bacteria culture were extracted following the method disclosed herein.

The primer sequences and the regions amplified are as follows. The underlined portions indicate primer binding regions:

Sequence: tcdA

| Name | Sequence |
|---|---|
| AF-1 | GCTACTAGAGGAAGAGATTCAAAATCCT |
| AR-C | ATCAACATCTAAATATACTCCGCCAAA |
| AF-2 | 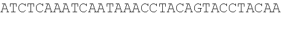 |

PCR DNA FRAGMENT length: 400 bp, 292 bp
GCTACTAGAGGAAGAGATTCAAAATCCTCAATTTGATAATATGAAATTTTACAAAAAAAGGATGGAATTTA
TATATGATA

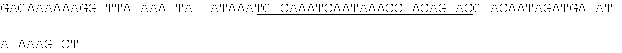

CATCTAGTATCTGAATATAATAGAGATGAAACTGTATTAGAATCATATAGAACAAATTCTTTGAGAAAAAT
AAATAGTAA
TCATGGGATAGATATCAGGGCTAATAGTTTGTTTACAGAACAAGAGTTATTAAATATTTATAGTCAGGAGT
TGTTAAATC
GTGGAAATTTAGCTGCAGCATCTGACATAGTAAGATTATTAGCCCTAAAAAATTTTGGCGGAGTATATTTA
GATGTTGAT
Sequence: tcdB

| Name | Sequence |
|---|---|
| BF-1 | CAGATGCAGCCAAAGTTGTTG |
| BR-C | TGTTGCCTTATCTCGAAGTACAAGTT |
| BF-2 | 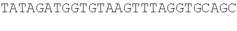 |

PCR DNA FRAGMENT length: 350 bp, 250 bp
CAGATGCAGCCAAAGTTGTTGAATTAGTATCAACTGCATTAGATGAAACTATAGACTTACTTCCTACATTA
TCTGAAGGA

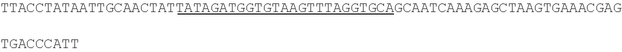

ATTAAGACAAGAAATAGAAGCTAAGATAGGTATAATGGCAGTAAATTTAACAACAGCTACAACTGCAATCA
TTACTTCAT
CTTTGGGGATAGCTAGTGGATTTAGTATACTTTTAGTTCCTTTAGCAGGAATTTCAGCAGGTATACCAAGC
TTAGTAAAC
AATGAACTTGTACTTCGAGATAAGGCAACA
Sequence: tcdC

| Name | Sequence |
|---|---|
| CF-1 | TTAGGCGTGTTTTTTGGCAAT |
| CR-C | CATATCCTTTCTTCTCCTCTTCTTCTTT |
| CF-2 | 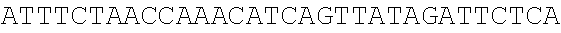 |

PCR DNA FRAGMENT length: 280 bp, 220 bp

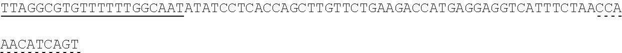

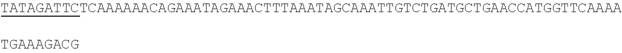

ACGAAAAGAAAGCTATTGAAGCTGAAAATCAACGTAAAGCTGAAGAAGCTAAAAAAGCTGAAGAAGCTAAA
AAGGCTGAA
GAACAACGCAAAAAAGAAGAAGAGGAGAAGAAAGGATATG
Sequence: edu2

| Name | Sequence |
|---|---|
| UF-1 | CAGCAGTCATGGGTGCAGTAGTAG |
| UR-C | AGCTGATATACTATTTCCTTGTGCCAAT |
| UF-2 | 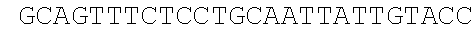 |

PCR DNA FRAGMENT length: 200 bp, 174 bp

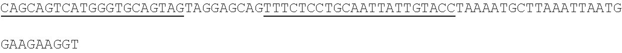

TATGGTACAGAAAAAAGTATTCCACAGATGCTTTTAGCAGGAACTTCTATAGACGATATTTTCGTAATTGT
GATGTTTAC
AGTATTCACAGGATTGGCACAAGGAAATAGTATATCAGCT
Sequence: tcdD

| Name | Sequence |
|---|---|
| DF-C | TGAATTAATTGTTTTAGCAAGAAATAACTCAGTAG |
| DR-1 | AAAAATATTATTAAATCTGTTTCTCCCTCTTCAT |
| DR-2 | TTAAATCTGTTTCTCCCTCTTCATAATGTAAA |

Comments: 135 bp, 125 bp
TGAATTAATTGTTTTAGCAAGAAATAACTCAGTAGATGATTTGCAAGAAATTTTATTTATGTTTAAGCCAT
TAGTAAAAA

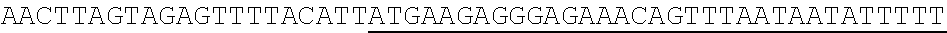

Reverse complement:

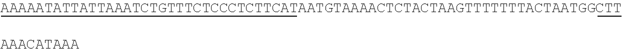

TAAAATTTCTTGCAAATCATCTACTGAGTTATTTCTTGCTAAAACAATTAATTCA

1. A method of detecting Clostridium difficile in a specimen, the method comprising determining the presence of one or more nucleic acid fragments representing a plurality of loci selected from the group consisting of nucleic acid regions whose sequences are designated as tcdA, tcdB, tcdC, tcdD and edu2.

2. The method of claim 1, wherein the loci tested is at least one of tcdC, tcdD and edu2.

3. The method of claim 1, wherein the presence of the nucleic acid fragments are determined by amplification of the plurality of loci by a plurality of primers selected from the group consisting of nucleic acid sequences:
AF-1
(SEQ ID NO: 1)
GCTACTAGAGGAAGAGATTCAAAATCCT
AR-C
(SEQ ID NO: 2)
ATCAACATCTAAATATACTCCGCCAAA
AF-2
(SEQ ID NO: 3)
ATCTCAAATCAATAAACCTACAGTACCTACAA
BF-1
(SEQ ID NO: 4)
CAGATGCAGCCAAAGTTGTTG
BR-C
(SEQ ID NO: 5)
TGTTGCCTTATCTCGAAGTACAAGTT
BF-2
(SEQ ID NO: 6)
TATAGATGGTGTAAGTTTAGGTGCAGC
CF-1
(SEQ ID NO: 7)
TTAGGCGTGTTTTTTGGCAAT
CR-C
(SEQ ID NO: 8)
CATATCCTTTCTTCTCCTCTTCTTCTTT
CF-2
(SEQ ID NO: 9)
ATTTCTAACCAAACATCAGTTATAGATTCTCA
UF-1
(SEQ ID NO: 10)
CAGCAGTCATGGGTGCAGTAGTAG
UR-C
(SEQ ID NO: 11)
AGCTGATATACTATTTCCTTGTGCCAAT
UF-2
(SEQ ID NO: 12)
GCAGTTTCTCCTGCAATTATTGTACC
DF-C
(SEQ ID NO: 13)
TGAATTAATTGTTTTAGCAAGAAATAACTCAGTAG
DR-1
(SEQ ID NO: 14)
AAAAATATTATTAAATCTGTTTCTCCCTCTTCAT
DR-2
(SEQ ID NO: 15)
TTAAATCTGTTTCTCCCTCTTCATAATGTAAA

4. The method of claim 1, wherein the presence of the nucleic acid fragments is determined by amplification of the plurality of loci representing at least two independent loci of C. difficile.

5. The method of claim 3, wherein the nucleic acid fragments representing each loci are amplified by one forward and two reverse primers, wherein one of the reverse primers is internal to the other reverse primer.

6. The method of claim 1, wherein the specimen is selected from the group consisting of a tissue biopsy, stool, swab, and bodily fluids.

7. The method of claim 1, wherein the specimen is isolated DNA.

8. The method of claim 3, wherein the amplification is performed directly on the specimen.

9. The method of claim 1, wherein the presence of one or more nucleic acid fragments representing the plurality of loci is determined by hybridization with one or more probes.

10. The method of claim 3, wherein the nucleic acid fragments are simultaneously amplified.

11. A method of detecting Clostridium difficile in a specimen, the method comprising:
(a) performing a multiplex polymerase chain reaction, wherein a plurality of nucleic acid fragments representing a plurality of loci in the bacteria are amplified by a plurality of forward and reverse primers, wherein for each pair of forward and reverse primer, a primer is internal, wherein the loci are designated tcdA, tcdB, tcdC, tcdD and edu2; and
(b) determining that the specimen is positive for the bacteria if a number of amplified nucleic acid fragments is sufficient to detect the bacteria in the specimen.

12. The method of claim 1, wherein the plurality of loci is at least one of tcdC, tcdD and edu2.

13. The method of claim 1, wherein the plurality of primers is selected from the group consisting of primers whose DNA sequences are:
AF-1
(SEQ ID NO: 1)
GCTACTAGAGGAAGAGATTCAAAATCCT
AR-C
(SEQ ID NO: 2)
ATCAACATCTAAATATACTCCGCCAAA
AF-2
(SEQ ID NO: 3)
ATCTCAAATCAATAAACCTACAGTACCTACAA
BF-1
(SEQ ID NO: 4)
CAGATGCAGCCAAAGTTGTTG
BR-C
(SEQ ID NO: 5)
TGTTGCCTTATCTCGAAGTACAAGTT
BF-2
(SEQ ID NO: 6)
TATAGATGGTGTAAGTTTAGGTGCAGC
CF-1
(SEQ ID NO: 7)
TTAGGCGTGTTTTTTGGCAAT
CR-C
(SEQ ID NO: 8)
CATATCCTTTCTTCTCCTCTTCTTCTTT
CF-2
(SEQ ID NO: 9)
ATTTCTAACCAAACATCAGTTATAGATTCTCA
UF-1
(SEQ ID NO: 10)
CAGCAGTCATGGGTGCAGTAGTAG
UR-C
(SEQ ID NO: 11)
AGCTGATATACTATTTCCTTGTGCCAAT
UF-2
(SEQ ID NO: 12)
GCAGTTTCTCCTGCAATTATTGTACC
DF-C
(SEQ ID NO: 13)
TGAATTAATTGTTTTAGCAAGAAATAACTCAGTAG
DR-1
(SEQ ID NO: 14)
AAAAATATTATTAAATCTGTTTCTCCCTCTTCAT
DR-2
(SEQ ID NO: 15)
TTAAATCTGTTTCTCCCTCTTCATAATGTAAA

14. A method for detecting toxigenic and non-toxigenic strains of Clostridium difficile in a specimen, the method comprising:
(a) performing a multiplex polymerase chain reaction, wherein a plurality of primers amplify a plurality of Clostridium difficile nucleic acid fragments selected from the group consisting of nucleic acid molecules whose sequences are designated tcdA, tcdB, tcdC, tcdD and edu2; and
(b) determining that the specimen is positive for toxigenic Clostridium difficile if a number of amplified fragments is sufficient to detect the bacteria in the specimen.

15. A primer or a probe to detect C. difficile, the primer or probe selected from the group consisting of nucleic acid molecules with sequences:
AF-1
(SEQ ID NO: 1)
GCTACTAGAGGAAGAGATTCAAAATCCT
AR-C
(SEQ ID NO: 2)
ATCAACATCTAAATATACTCCGCCAAA
AF-2
(SEQ ID NO: 3)
ATCTCAAATCAATAAACCTACAGTACCTACAA
BF-1
(SEQ ID NO: 4)
CAGATGCAGCCAAAGTTGTTG
BR-C
(SEQ ID NO: 5)
TGTTGCCTTATCTCGAAGTACAAGTT
BF-2
(SEQ ID NO: 6)
TATAGATGGTGTAAGTTTAGGTGCAGC
CF-1
(SEQ ID NO: 7)
TTAGGCGTGTTTTTTGGCAAT
CR-C
(SEQ ID NO: 8)
CATATCCTTTCTTCTCCTCTTCTTCTTT
CF-2
(SEQ ID NO: 9)
ATTTCTAACCAAACATCAGTTATAGATTCTCA
UF-1
(SEQ ID NO: 10)
CAGCAGTCATGGGTGCAGTAGTAG
UR-C
(SEQ ID NO: 11)
AGCTGATATACTATTTCCTTGTGCCAAT
UF-2
(SEQ ID NO: 12)
GCAGTTTCTCCTGCAATTATTGTACC
DF-C
(SEQ ID NO: 13)
TGAATTAATTGTTTTAGCAAGAAATAACTCAGTAG
DR-1
(SEQ ID NO: 14)
AAAAATATTATTAAATCTGTTTCTCCCTCTTCAT
DR-2
(SEQ ID NO: 15)
TTAAATCTGTTTCTCCCTCTTCATAATGTAAA
